(19) United States
(12) Patent Application Publication
(10) Pub. No.: US 2026/0358002 A1
(43) Pub. Date: Nov. 12, 2026

(54) Modular Ventilation System with Environmental Sensing and Dehumidification

(71) Applicant: Stephen Albert CHIN YEE, Englewood, FL (US)

(72) Inventor: Stephen Albert CHIN YEE, Englewood, FL (US)

(21) Appl. No.: 19/421,084

(22) Filed: Dec. 16, 2025

Related U.S. Application Data
(60) Provisional application No. 63/804,154, filed on May 12, 2025.

(51) Int. Cl. F24F 3/16; A61L 9/20; B01D 46/00; B01D 46/62; B01D 53/00; B01D 53/04; B01D 53/26; B01D 53/30; B01D 53/34; B01D 53/88; F24F 3/14; F24F 8/108; F24F 8/158; F24F 8/167; F24F 8/22; F24F 11/38; F24F 11/39; F24F 11/64; F24F 110/12; F24F 110/22; F24F 110/64; F24F 110/70; F24F 120/10

(52) U.S. Cl. CPC F24F 3/16; A61L 9/20; A61L 9/205; B01D 46/0006; B01D 46/0013; B01D 46/0028; B01D 46/0036; B01D 46/0038; B01D 46/62; B01D 53/007; B01D 53/0407; B01D 53/265; B01D 53/30; B01D 53/346; B01D 53/885; F24F 3/1405; F24F 8/108; F24F 8/158; F24F 8/167; F24F 8/22; F24F 11/38; F24F 11/39; F24F 11/64; A61L 2209/111; A61L 2209/12; A61L 2209/14; A61L 2209/16; B01D 2253/102; B01D 2255/20707; B01D 2255/802; B01D 2257/708; B01D 2257/80; B01D 2257/90; B01D 2258/06; B01D 2259/4508; B01D 2259/804; B01D 2267/40; B01D 2279/50; F24F 2110/12; F24F 2110/22; F24F 2110/64; F24F 2110/70; F24F 2120/10

(57) ABSTRACT

A modular ventilation system integrates an H-Cell dehumidifier module, multi-stage MERV-13 filtration, UV-C germicidal irradiation, and photocatalytic oxidation (PCO) within a reversible-airflow cabinet for residential and light commercial applications. The system features motorized dampers controlling outside and recirculated air based on real-time environmental sensors monitoring temperature, humidity, CO₂, and PM2.5 levels. A central PCB executes three operational modes: automatic hourly cycling, manual app-based control, and cloud-connected predictive analytics. The H-Cell dehumidifier module incorporates quick-disconnect connectors for tool-free replacement. Service-friendly features include removable PCB holders, LED status indicators, and QR-coded components. The system scales from 700 to 2,200 square feet of conditioned area through dip-switch configuration and improves indoor air quality of within the conditioned space through dynamic damper modulation and multi-stage filtration processes, acting in part or whole as an integrated system.

### CROSS-REFERENCE TO RELATED APPLICATIONS

Claims Priority of Provisional filed dated 2025 May 12 with patent No. 63/804,154.

Claiming priority to earlier provisional patent application.

The following specification particularly describes the invention and the manner in which it is to be performed.

### BACKGROUND OF THE INVENTION

### Field of the Invention

The present invention relates to residential and light commercial indoor environmental systems, ventilation, indoor air purification, and dehumidification systems. More specifically, it pertains to a Modular Ventilation System with Environmental Sensing and Dehumidification designed for homes and small buildings. This smart ventilator system intelligently manages ventilation, humidity, and air quality through realtime air quality monitoring and predictive control and filtration capabilities to dynamically manage airflow, humidity, and pollutant levels. The present invention incorporates real-time environmental sensing, dehumidification, and adaptive air quality management in an integrated and serviceable housing.

### Background of the Invention

Conventional residential ventilation and dehumidification systems suffer from a number of limitations. Current indoor air quality and humidity management systems often consist of separate, uncoordinated devices—such as dehumidifiers, motorized dampers, air filters, fans, and smart controllers—which typically require complicated setup and lack seamless integration. These systems tend to operate independently or do not follow flexible control protocols, making them ineffective at adapting to changing environmental conditions both indoors and outdoors. Conventional indoor air management systems often utilize multiple, non-integrated devices—dehumidifiers, air purifiers, fans, and dampers—resulting in inefficient operation, complex installations, and suboptimal environmental control. These systems typically lack humidity-aware ventilation, modular scalability, predictive air source selection, and integrated sensor-driven automation.

Prior art disclosed in Commercial systems such as Honeywell TrueFRESH™, but do not include onboard dehumidification or predictive control algorithms responsive to indoor and outdoor air quality conditions.

Specifically, the limitations of current systems include:
Lack of humidity-aware ventilation: Fresh air intake systems often bring outside air without evaluating humidity or dew point, leading to indoor discomfort and potential Mold risk.
Inefficient energy use: Most systems do not consider external humidity, temperature, pollution levels, or cycling the central Air Conditioner off during favourable climate conditions, leading to excessive HVAC load and introduction of pollutants such as smoke and pollen into the controlled environment.
Absence of modularity: Dehumidifiers, air filters, and ventilators are not integrated into a single customizable unit, requiring complex installations and need separate maintenance.
Lack of responsive ventilation rates: Existing systems typically rely on static airflow rates, leading to over-ventilation during unoccupied periods and under-ventilation during high occupancy or pollutant spikes.
Limited sensor integration: Most residential devices do not incorporate multi-parameter environmental sensing (e.g., CO₂, VOCs, PM2.5), which restricts their ability to respond to comprehensive indoor air quality (IAQ) metrics.
No intelligent air source switching: Existing ventilation systems typically lack integration with motorized dampers or environmental sensors capable of selecting the optimal inlet air source based on dynamic environmental data.
Lack flexibility for left-right air flow reconfiguration based on installation constraints.

The existing systems lacks integration of motorized dampers that would intelligently control and select the optimal inlet air source based on environmental conditions.

Thus, there is a need for an integrated, modular, intelligent Ventilation System with Environmental Sensing and Dehumidification that can determine the best inlet air source to adaptively manage ventilation and humidity while filtering and sanitizing indoor air based on real-time IAQ data and predictive environmental modelling.

### SUMMARY OF THE INVENTION

The present invention provides a modular smart ventilation system designed to improve indoor air quality, manage humidity levels, and reduce energy consumption in residential and light commercial environments. The system of the present invention integrates Modulating dampers, air filtration, controlled ventilation, active dehumidification, UV sanitization, and intelligent sensor based operation within a compact cabinet structure. The main components of the present system includes: a modular H-Cell dehumidifier with refrigerant-based moisture removal, dual condensate pans, and a serviceable compressor section; Multi-stage filtration with tool-free access, including MERV 13 and activated carbon filters; Dynamic airflow control using dampers for return and outside air, governed by real-time humidity and temperature sensing; UV sanitization of the air and evaporator surface to reduce pathogens; Intelligent control via a dedicated PCB, with service-friendly design including removable holders, service ports, and LED status indicators; Optional air bypass venting and secondary condensate base pan and safety features like overflow switches. The present invention is a robust, energy-efficient system with modularity, allowing easy maintenance, upgrades, and adaptability to various air quality demands. This system provides an efficient, serviceable, and intelligent solution for maintaining healthy indoor environments while minimizing energy usage and maximizing operational reliability.

The system comprises a cabinet housing that supports multiple removable modules including:
An H-Cell dehumidifier with dual condensate pans and refrigeration cycle
Multi-stage air filtration units with tool-free access
UV sanitization
Motorized dampers for fresh and return air management
A smart PCB controller connected to environmental sensors (e.g., temperature and humidity)

The system supports three operating modes: Default (automated), Manual (user app control), and Smart (cloud-connected with predictive control and analytics). It is designed to be scalable, serviceable, and energy-efficient.

### BRIEF DESCRIPTION OF THE DRAWINGS

FIG. 1 is an exploded perspective isometric view of Core Module in Right air Flow Configuration of the modular dehumidifying smart ventilator system, illustrating the main housing and modular components including ventilation, dehumidification, and air filtration modules.

FIG. 2 is a Core Module in Left air Flow illustrating internal component placement including airflow pathways, environmental sensors, condenser coil assemblies, and access compartments for filtration and electrical connections.

FIG. 3 is a detailed structural view of the H-Cell dehumidification module and it's required self contained components, consisting of base frame, refrigeration coils, compressor, drain pans, control, drain outlet and safety switches.

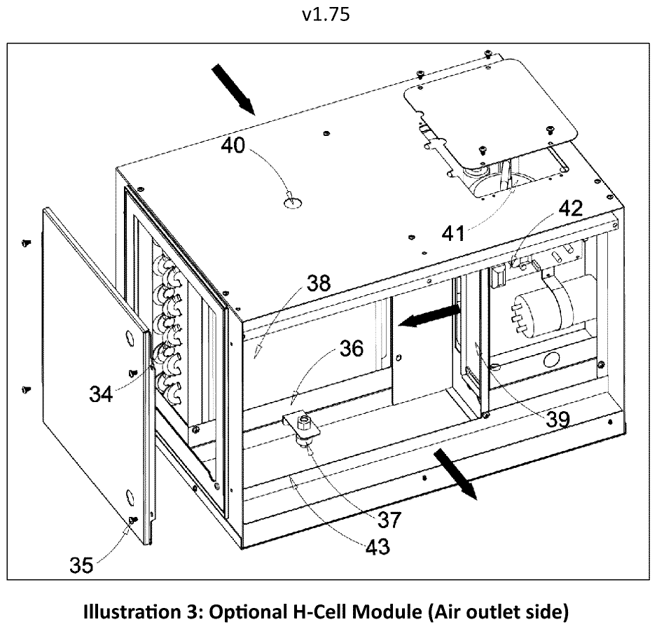

FIG. 4 is a schematic installation diagram illustrating the placement of the ventilation system within a residential or light commercial attic space, showing duct connections for outdoor air intake, indoor return air, treated air supply, and smart control placement.

### DETAILED DESCRIPTION OF THE INVENTION:

The present invention provides a modular Ventilation System with Environmental Sensing and Dehumidification that combines multiple environmental control functions into a single, integrated solution. The system adapts to varying conditions by dynamically regulating ventilation, humidity, and air quality, thus optimizing the indoor environment for both comfort and energy efficiency.

In one embodiment, Modular Housing and Configuration of the modular dehumidifying smart ventilator system is disclosed. The system is housed within a modular enclosure, designed to accommodate interchangeable components including a ventilation module, dehumidification module, and air filtration module. Each module is designed to be independently replaceable or upgradeable, ensuring ease of maintenance and the ability to customize the system based on specific user needs.

In another embodiment, the top and bottom cabinets contain essential control components, including the Control PCB, air inlet motorized dampers, sensors, filter, air cleaners, fan assembly, UV sanitizing features, H-Cell EZ electric plug, removable PCB holder, LED port label, and the electrical cover, all shielded with cabinet insulation to enhance efficiency. Both cabinets function identically and allow the entire enclosure to be rotated 180 degrees to allow the installation horizontal left or right based on the installer preferences, sharing the characteristics to support the heavier mechanical components within the H-Cell (dehumidifier).

The H-Cell module is central to the dehumidification process, comprising an evaporator coil for moisture condensation, a condenser coil for heat dissipation, a compressor for refrigerant circulation, and a condensate collection and drainage mechanism. The UV sanitizer bulb and port enable microbial sterilization of the evaporator coil and air path, enhancing hygiene.

Air is drawn into the system by a fan secured by a fan bracket and powered by a high-voltage power cable. A series of filters, including a pre-filter, MERV 13 filter, and activated carbon filter, ensures thorough particle and gas removal. The airflow is further regulated by return air and outside air dampers, each equipped with dedicated service access for motor maintenance.

Yet in another embodiment, a network of sensors, particularly the humidity/temperature sensor, monitors incoming air conditions and feeds data to the control logic on the PCB. The system uses this input to adjust airflow rates, damper positions, and H-Cell operation dynamically for energy-efficient and responsive environmental management. Maintenance is simplified through toolless filter access, removable PCB holders, service covers, and a tool-free filter access mechanism comprising hinges and a toggle latch. Adjustable air bypass vents allow airflow fine-tuning and secure component mounting. The modular design enables optional upgrades or replacements, such as the H-Cell module, without requiring full system disassembly.

In another embodiment, The Modular Dehumidifying Smart Ventilator System comprising of: A modular H-Cell dehumidifier comprising an evaporator coil, condenser coil, compressor, and dual condensate pans for efficient moisture removal; Multi-stage filtration, including a pre-filter, MERV 13 filter, and activated carbon filter, with toolless access for easy maintenance. Dynamic air intake and recirculation management through electronically controlled outside and return air dampers, equipped with service access panels; A UV sanitization unit integrated into the air path and evaporator region to kill airborne pathogens; A Control PCB and associated logic that monitors and adjusts system behaviour based on inputs from a humidity and temperature sensor; and Service-friendly design features such as removable PCB holders, tool-free filter access mechanisms with hinges and toggle latches, LED port labels, strain relief cable locks, and dedicated access ports. Optional airflow bypass vent and overflow detection system for enhanced reliability and safety.

These design elements support quick installation, easy maintenance, and adaptability to varying environmental loads or infrastructure types.

In another embodiment, Dynamic Air Intake and Recirculation Management unit is disclosed. The system features electronically controlled air dampers that manage the intake of outside air, and the flow of return air from the conditioned space. These dampers regulate the proportions of fresh and recirculated air entering the system. Each damper is paired with dedicated service access panels, allowing easy inspection, motor maintenance without disassembling other parts of the unit.

In another embodiment, Control PCB unit with Environmental Sensor Feedback is disclosed. A central Control Printed Circuit Board (PCB) governs the operation of all active components including the fan, H-Cell compressor, dampers, and UV light. It processes input from an integrated humidity and temperature sensors, which continuously measures the environmental conditions of incoming air. Based on real-time sensor data, the PCB dynamically adjusts system performance to optimize humidity control, ventilation rates.

In another embodiment, Installation and Service-Friendly Design Features are disclosed. The system is engineered for ease of access and maintenance, incorporating:
A removable PCB holder, which allows technicians to access components below the control board.
Tool-free filter access mechanisms comprising hinges and toggle latches on filter covers and access panels for tool-free servicing.
LED port labels on the electrical cover, providing visual confirmation of operational status through integrated indicator lights.
Cable bracket and cable mounting system for a convenient method to install and suspend the ventilator system.

In a preferred embodiment, the H-Cell dehumidifier module incorporates quick-disconnect High and low voltage plug for the power and controls, allowing the H-Cell to be replaced or serviced with ease. The system optionally employs machine learning algorithms to analyze historical usage patterns and optimize operation cycles. Additional sensors may include Humidity, PM2.5 particulate matter, CO2, VOC, and Temperature sensors for comprehensive air quality monitoring. Service components include QR codes that link to digital documentation and service histories stored in cloud databases.

Dedicated access ports and service covers for the H-Cell, damper motors, and electrical sections, minimizing downtime during inspection or replacement procedures.

In another embodiment, Optional Airflow Bypass and Overflow Protection unit is disclosed. The system includes: An adjustable bypass air vent that allows a portion of airflow to bypass the H-Cell coils under certain conditions, maintaining optimal flow while improving the moisture removal efficiency, and a condensate overflow detection switch integrated into the secondary pan, which triggers a system shutdown or alert if water levels exceed safe thresholds, preventing internal and external water damage or hazardous conditions.

In another embodiment, the system is configured to prioritize optimal indoor air quality by maintaining appropriate relative humidity levels while operating with maximum energy efficiency. The control unit intelligently manages the operation of each component by utilizing real-time environmental data combined with predictive algorithms. This approach ensures that energy is consumed only when necessary, thereby reducing overall operational costs without compromising occupant comfort.

The scalable, energy-efficient IAQ (Indoor Air Quality) optimization is achieved by incorporating all system components into a single, easy-to-understand cabinet engineered with plug-and-play conformity in mind. This all-in-one design streamlines installation, simplifies user interaction, and minimizes potential errors during setup or service.

The ventilator unit is designed for flexible installation across a broad range of home sizes—from as small as 700 square feet to homes up to 2,200 square feet in living area. For larger residences or commercial buildings, multiple ventilator units may be installed to meet greater airflow and dehumidification demands. The system's scalability is enabled by a configurable setting on the control unit's printed circuit board (PCB), which is adjusted during installation. This setting calibrates the ventilator's runtime, allowing it to deliver the proper volume of fresh air and moisture control tailored to the specific size and requirements of the conditioned space.

In another embodiment, the invention Default Logic Mode with All-In-One Smart ventilation and dehumidification system comprising of Modules controlled by the Sensor Array and Control:
Sensor Array and Control: Sensors are connected to a central microcontroller or SoC with real-time processing capability.
The control logic executes in the following order: Control references its dip switch setting the installer selected, per the home's preset ventilation requirements.
Read sensor data at 20-second intervals.
Compares the entering outdoor air parameters against user-defined comfort thresholds.
Predict IAQ degradation or humidity spikes based on historical trends.
Adjust airflow run time, dampers, dehumidifier activation, and purification based on optimization matrix.

In another embodiment, the system optionally includes a Photocatalytic Oxidation (PCO) air sanitizing module positioned downstream of the fan within the electrical section, serving as a final-stage air purifier. The PCO module utilizes ultraviolet (UV) light in combination with a photocatalytic substrate, typically titanium dioxide (TiO₂), to break down pollutants at the molecular level. Unlike traditional filtration which relies on physically capturing particles, PCO technology actively destroys contaminants through oxidation reactions. When UV light strikes the catalyst surface, it creates hydroxyl radicals and super-oxide ions that decompose volatile organic compounds (VOCs), odors, bacteria, and viruses into harmless by products such as carbon dioxide and water vapor. The PCO module housing is configured for in-line duct mounting and includes electrical interface connectors for integration with the main control PCB, allowing coordinated operation with other system components. The module incorporates safety features including an interlock switch that prevents UV operation when the housing is opened, and an hours-run meter for tracking UV lamp usage and scheduling replacement.

The system of the present invention operates through three (3) Operation Modes:
1. Default Mode: (No external sensor or input required)
Fully automatic operation based on initial air flow setting for the controlled environment fresh air requirements, central microcontroller or SoC controller programmed algorithm, based on the fresh air Humidity and Temperature metrics.
Default Mode Logic: Every Hour the Controller cycles the Fan and fresh air damper open, and after a predetermined time, the controller dynamically decides whether to turn on the H-Cell to dry the incoming outdoor air based on the entering Relative Humidity. After the original ventilation fan cycle time has expired the Smart Ventilation Control Logic determine if the system will need to additionally run time to decrease the space's Relative Humidity. In this phase the Fan, H-Cell, and recirculation air damper will be opened, to additionally dry out and clean the air in the conditioned space. The processor controller will repeat these cycles every hour, without any external sensor or commands.
b. Default Mode Module components:
   Fan: An electrical motorize device used to facilitate the air movement through the ventilator to conditioned space.
   H-Cell Modular Dehumidifier: A pluggable dehumidification module using a refrigerant cycle, with slide-in installation and autodetection by the system controller.
   Multi-Stage Air Filtration Unit: Three stage filter air cleaner system compromised of Pre-filter, Activated carbon filter, & MERV filter
   Smart Predictive Control System: Smart PCB controllers that Anticipates ventilation and dehumidification needs, adjusts damper positions, fan & cycle timing.
   Motorized Dampers: Outdoor air damper and indoor recirculation
Air Sanitizer: UV-C or photocatalytic oxidation (PCO) purifier module.
2. Manual Mode: (Include Default Mode Features. Requires-External Monitor, Wi-Fi & App).
   App: Smart Device App enabled with Location Wi-Fi.
   Quick Vent: Immediate user uses the app to-triggered the controller to open the fresh air damper to open, and run the fan for a boost of fresh air and 3 stage filtration for 05- 10- 59 minutes.
   Monitor: View Space Temperature, Humidity, Co2, VOC & Dust levels.
   Vacation setting: Via the app Limit the operation while the occupants are away.
   Enhancements: Smart device App includes history, notification, firmware updates via Wi-Fi.
   Fail-safe Default Mode: If the inside IAQ monitor were to fail or loose communication with the wifi, the system will revert to Default Mode to protect the home's indoor environment.
3. Smart Mode: (Same as Manual Mode Features and Requirements, Subscription Required).
   IAQ Focus setting: Good, Better, & Best settings, Demand control of Indoor Air Quality components within the invention of the devices based on actual inside conditions.
   Automated Efficiency setting: Limits operation of the central Air Conditioning when there is an advantage to use the outside air to cool the living space.
   Access to Personal Dashboard: Maintenance logs tracked over cloud for professional servicing, site analytics, reports, uploads, Email notifications.
Fail-safe Default Mode: If the inside IAQ monitor or Wi-Fi were to fail, the system will revert to Default Mode to protect the home's indoor environment.

System Housing and Architecture: The primary system housing (“core unit”) is a powder-coated, 18-gauge metal enclosure with thermally and acoustically insulated inner walls. It contains:
Main centrifugal fan (UV rated Backward Incline UV rated fan, efficient and reliable PSC motor, low noise bearing).
Integrated control board with microprocessor, Wi-Fi, and Bluetooth option.
Inlet duct collar for fresh air and return air.
Outlet collar for conditioned air supply.
Quick-lock mounting for the H-Cell and filtration modules

Power and Compliance:
Standard operation at 120V, 60 Hz, single phase.
Thermal overload protection for motor.
UL 507 and exceeds ASHRAE 62.2 compliance.
Fire-rated insulation (UL94 HB).
Built-in diagnostic LEDs for installation feedback.

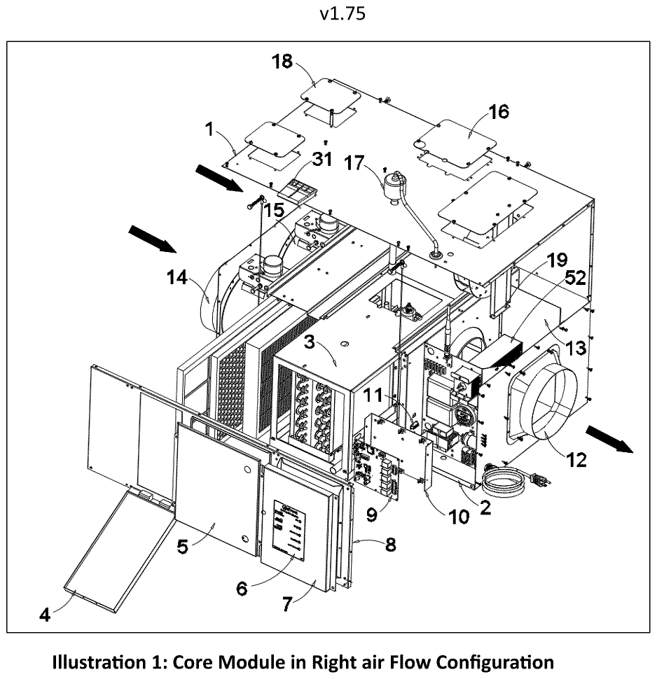

In another embodiment, FIG. 1 is an exploded isometric view of Core Module in Right air Flow Configuration. Here's a breakdown of the major visible components and features in the image:
Top Cabinet (1): The upper part of the cabinet structure (1) houses various system components, providing protection and support to the internal mechanisms.
Bottom Cabinet (2): The lower portion of the cabinet structure (2), completes the housing when fastened the Top Cabinet ensuring overall stability and support for the system.
H-Cell (Dehumidifier) (3): H-Cell (Dehumidifier)(3) is a specialized component that helps remove moisture from the air, controlling humidity levels and enhancing air quality by preventing Mold and mildew growth.
Toolless Filter Access (4): Toolless Filter Access (4) is a feature that allows easy removal and replacement of air filters without the need for tools, enhancing convenience and ease of maintenance.
H-Cell Service Cover (5): A service access cover (5) for the H-Cell, enabling quick access of refrigeration ports, and electrical without removing the entire assembly.
LED Port label (6): A LED Port label (6) is affixed to the electrical cover to allow for visual confirmation of the ventilator status via the LED indicators on the Hub Control PCB.
Electrical cover (7): A protective cover (7) that encloses electrical components, preventing dust, moisture, or other contaminants from damaging the system's wiring and circuit boards.
Cabinet Insulation(8): Insulating material placed inside the unit to reduce energy loss, improve efficiency, reduces component noise, and control temperature regulation within the system.
Control PCB(9): The control board (9) that regulates the operation of the system, managing the various components such as Fan, H-Cell, Dampers and other electronics.
Removable PCB Holder(10): A detachable holder (10) designed to securely support the control PCB, allowing for easy removal or replacement of components beneath it during servicing.
H-Cell EZ Electric Plug(11): H-Cell EZ Electric Plug(11) is a user-friendly electrical plug designed for the H-Cell, allowing easy connection and disconnection for maintenance or replacement.
Outlet air funnel(12): Outlet air funnel(12) is a funnel that directs the treated and filtered air from the system to the desired outlet, ensuring smooth airflow and preventing air turbulence.
Air Guide(13): Air Guide(13) is a component designed to direct airflow within the system, improving air distribution and ensuring efficient performance.
Damper Return Air(14): A damper (14)that controls the flow of return air back into the system. It regulates the recirculated air from the conditioned space into the ventilator and ensures proper filtration, dehumidification, and sanitizing of the air.
Damper Outside Air(15): A damper (15) controls the intake of outside air into the system, introducing less polluted air into space, and ensures proper filtration, dehumidification, and sanitizing of the air.
H-Cell Service Cover(16): H-Cell Service Cover(16) is a service access cover for the H-Cell, enabling quick access of refrigeration ports, and electrical without removing the entire assembly.
UV Sanitizer bulb(17): UV Sanitizer bulb(17) is a specialized ultraviolet light bulb designed to sanitize and disinfect the Evaporator coil and air as it passes through the system, killing harmful bacteria, viruses, and other pathogens.
Damper Motor Service Access(18): A designated access panel (18) allows maintenance and servicing of the damper motor. This feature ensures that users can easily perform maintenance tasks without requiring disassembly of the entire system.
Bracket Pegs(19): Small pegs (19) are used to hold fan brackets in place.

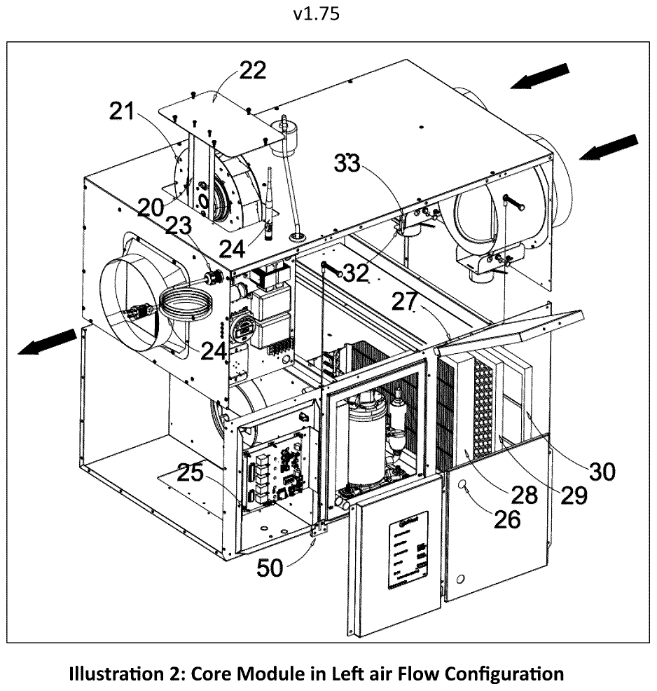

In another embodiment, FIG. 2 illustrates Core Module in Left air Flow.
Bracket Holder/Cover(20): Bracket Holder/Cover(20) is a cover designed to hold the bracket in place, typically offering both support and cover, ensuring secure positioning within the system and easy replacement of the Fan/Motor assembly.
Fan(21): The fan (21) is responsible for drawing air into the system, initiating airflow and ensuring proper circulation of air within the unit.
Fan Bracket (22): Fan Bracket (22) is a supporting bracket for securing the fan in position within the system to prevent movement during operation and ensure efficient airflow.
Cable Strain Relief (23): Cable Strain Relief (23) “locks” into place, absorbing any tension from outside pulls so the cable connection inside stays protected.
Power Cable(24): Power Cable(24) delivers high voltage power to the fan motor & Transformer, typically connects the unit to your mains electricity with 115V AC.
Dual locking circuit board support, type 1(25): a mechanical fastener used to securely mount a printed circuit board (PCB).
Plugs(26): Silicon or similar material to plug holes that aren't currently needed to prevent air being pulled through an open hole.
Tool-free Filter Access Door(27): Tool-free Filter Access Door(27) comprises hinges and a toggle latch mechanism that secures the filter cover, allowing easy removal or installation of the filter without requiring tools.
Merv 13 Filter(28): It is a high-performance filter rated MERV 13, capable of capturing fine particles such as dust, pollen, pet dander, and Mold spores for superior air quality.
Activated Carbon Filter(29): Activated Carbon Filter(29) is a filter designed to remove odours, gases, and volatile organic compounds (VOCs) from the air, improving air quality and freshness.
Pre Filter (30): Pre Filter (30) is a filter that captures larger particulates before air passes through more specialized filters, prolonging their lifespan and improving overall filtration efficiency.
Humidity Sensor/Temperature Bracket (32): Humidity Sensor/Temperature Bracket (32) physically mounts the IP65 humidity/temperature sensor inside the device or on an external surface, keeping it stable and secure.
Humidity/Temperature Sensor (33): It measures the relative humidity and temperature for the incoming outside air, as well as it helps the ventilator and H-Cell adjust airflow or operation automatically, depending on moisture levels.

In another embodiment, Optional H-Cell Module (Air outlet side) is shown in FIGS. 2 & 3.
Evaporator Coil(34): Evaporator Coil (34) is a coil used in the cooling process that absorbs heat from the air and turns refrigerant from a liquid to a gas, creating a cold surface to condensate the moisture out of the air before it is returned to the environment.
Cover Screws(35): Cover Screws(35) designed to secure the damper cover, protecting the damper components from external debris or damage while ensuring proper functioning.
Condensate Pan(36): Condensate Pan (36) is a pan with a drain outlet that collects condensate generated by the dehumidification process and providing a method to connect a drain tube or pipe so the water can be redirected to a safe place.
Condensate Overflow Switch (37): Condensate Overflow Switch (37) is a switch that detects when the condensate pan has overflowed, signalling the system to stop and activate an alarm to prevent water damage.
Condenser Coil(38): Condenser Coil (38) is a coil responsible for releasing heat from the refrigerant, causing it to change from a gas back to a liquid during the system's humidity removal process.
Bypass Air Adjustable Vent(39): Bypass Air Adjustable Vent (39) is a vent that allows air to bypass the H-Cell Coils for optimum dehumidification and maintaining the proper air flow through the system, under different conditions.
UV Sanitizer Port(40): UV Sanitizer Port(40) is a dedicated port or opening through which the UV sanitizer bulb is inserted, allowing it to emit UV light and disinfect coil surfaces and the air passing through.
H-Cell Compressor(41): H-Cell Compressor(41) is a compressor within the HCell that facilitates the dehumidification process by compressing and circulating refrigerant gas, aiding moisture removal.
H-Cell Electrical Section(42): The electrical components housed within the HCell, which include circuits and wiring, switches, motor capacitors, needed for powering and controlling the dehumidification process.
Base/Secondary Condensate Pan(43): A secondary condensate pan (43) is located at the base of the unit to catch overflow from the primary pan and prevent water from leaking outside the unit.

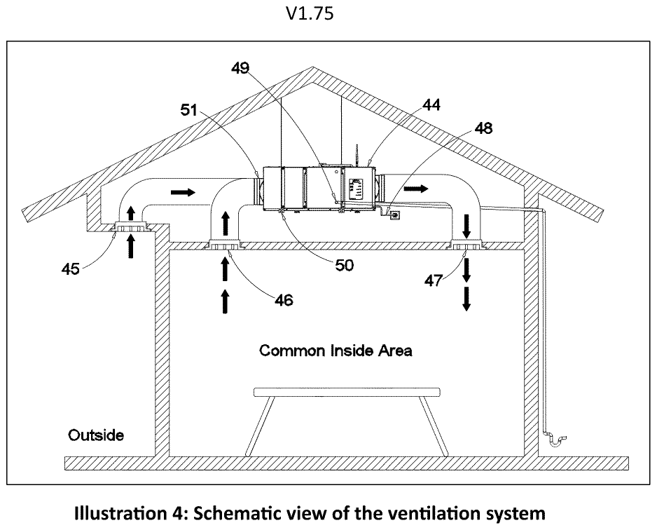

In another embodiment, A schematic view illustrating the ventilation system equipped with a H-Cell and typical duct connections according to the present invention is shown in FIG. 4.
Ventilator (44): The invention including all components in a standard installation's application.
Fresh Air Inlet (45): This is where outside air enters the system. It brings in fresh air to ventilate the house, improving air quality and oxygen levels indoors.
Recirculated Air Inlet (46): This inlet draws air from inside the house back into the system for dehumidification, filtering and reconditioning.
Clear Dry Supply Air (47): This is the output vent that delivers purified, conditioned (filtered and dehumidified) air back into living space.
Power Cord (48): Provides electrical power to the unit, allowing the Fan, HCell, Air sanitizers, Sensors, and other electrical components to operate.
Condensate Drain Outlet (49): Removes condensed moisture from the system, important in humid environments or when dehumidification occurs inside the system.
Bracket Cable Support (50): Provides a convenient method to suspend the ventilation, dehumidification system.
Damper Inlet (51): A movable flap that regulates airflow into the system, it is either open or close to control how much air comes in and is manipulated by smart ventilation controls.

The advantages of the present invention are as follows:
a. Improved ease of installation and enhanced reliability by integrating all major air quality improvement components—ventilation, dehumidification, variable inlet airflow, filtration, control, wiring, and air sanitization—into one compact, unified cabinet.
b. Improved energy efficiency through adaptive, inlet air integrated damper control for demand-based operation.
c. Enhanced comfort and well-being of occupants by maintaining optimal indoor air quality.
d. Effective control of humidity to prevent issues such as mold and mildew.
e. Allowance for limiting the operation of the central cooling system when outside conditions permit, resulting in meaningful energy savings without compromising indoor comfort.
f. Scalability and flexibility are afforded by modular architecture, allowing customization based on specific building needs.
g. Dynamic responsiveness to both indoor and outdoor environmental changes, enabling proactive system adjustments.

The invention comprises:
1. Modular Cabinet System:
An enclosure with top and bottom sections, thermally insulated, capable of left or right air flow configuration. Allows installation flexibility and houses all major subsystems.
2. H-Cell Dehumidifier Module:
Incorporates evaporator and condenser coils, compressor, dual pans, overflow switch, and control electronics. Detachable for easy service or upgrade.
3. Multi-Stage Air Filtration:
Pre-filter, MERV 13 filter, and activated carbon filters installed in a tool-free tray. Removes particulates, VOCs, and odours.
4. UV Air Sanitization:
A UV-C bulb sterilizes air passing over coils to eliminate microbial contaminants.
5. Environmental Sensors:
Monitors include humidity and temperature sensors mounted near the air inlets. Data is relayed to the PCB for control decisions.
6. Control PCB:
Central controller includes microprocessor with default logic and override via Wi-Fi app or subscription-based analytics dashboard. Controls fan, dampers, UV light, and dehumidifier.
7. Motorized Dampers:
Independently controlled dampers regulate outside air intake and indoor return air recirculation, responding to real-time sensor data.
8. Operation Modes:
   Default Mode: Hourly cycles based on factory-set logic.
   Manual Mode: User-app control and monitoring.
   Smart Mode: Predictive logic, cloud analytics, failover protections.
9. Service and Installation Features:
   Removable PCB holder.
   LED port labels.
   Toolless access.
   Cable strain reliefs.
   Adjustable bypass vents.
   Integrated Cable Mounting Brackets, providing for easier installation.
10. Scalability:
System can be calibrated for homes ranging from 700-2200 sq. ft., and scaled with multiple units for larger spaces.

What is claimed is:

1. A modular ventilation system with environmental sensing and dehumidification, comprising:
a thermally insulated cabinet having reversible mounting provisions for selective configuration between left-to-right and right-to-left airflow directions, said cabinet housing all major subsystems;
at least one fan configured to move air through said cabinet;
at least one removable dehumidifier module including refrigeration components comprising an evaporator coil, a condenser coil, and a compressor, said module further including dual condensate pans with overflow protection;
a multi-stage air filtration assembly comprising at least three distinct filtration elements mounted in a removable tray;
at least one UV-C germicidal irradiation (UVGI) unit positioned to irradiate air flowing through the system;
a damper system comprising motorized dampers configured to selectively control outside-air intake and indoor-air recirculation;
a sensor array comprising at least one humidity sensor and at least one temperature sensor; and
a control system configured to receive data from said sensor array and control operation of said fan, said dehumidifier module, said UVGI unit, and said damper system based on environmental conditions.

2. The modular ventilation system of claim 1, wherein said removable dehumidifier module comprises:
quick-disconnect high and low voltage electrical connections enabling hot-swap replacement without the need to disconnect inlet and outlet ducts directing air to and from a conditioned space;
integrated module-specific control electronics communicating with said control system; and
visual indicators displaying module operational status;
wherein said removable dehumidifier module is configured in an H-Cell arrangement with said evaporator coil and said condenser coil positioned in parallel vertical planes.

3. The modular ventilation system of claim 1, wherein said control system comprises a printed circuit board (PCB) assembly with modular architecture supporting field-programmable operation modes.

4. The modular ventilation system of claim 1, wherein said multi-stage air filtration assembly comprises:
a pre-filter rated for large particle removal;
a MERV-13 or higher rated filter for fine particle filtration; and
an activated carbon filter for odor and VOC reduction;
wherein all filters are housed in a sliding tray accessible without tools.

5. The modular ventilation system of claim 4, wherein said sliding tray includes individual filter presence sensors connected to said control system for filter monitoring and maintenance alerts.

6. The modular ventilation system of claim 1, wherein said UV-C germicidal irradiation (UVGI) unit comprises UV-C lamps positioned adjacent to said evaporator coil, said lamps having wavelengths between 254-265 nanometers optimized for microbial inactivation.

7. The modular ventilation system of claim 6, further comprising a secondary UV-C germicidal irradiation (UVGI) unit comprising a titanium dioxide (TiO₂) photocatalytic cell positioned downstream of said filtration assembly, said secondary UVGI unit creating a photocatalytic oxidation (PCO) system that neutralizes airborne pathogens, volatile organic compounds (VOCs), and odors for dual-stage air sanitization.

8. The modular ventilation system of claim 3, wherein said PCB assembly supports three distinct operational modes:
an automatic mode executing predetermined control algorithms based on sensor feedback;
a manual override mode enabling direct user control of individual system components; and
an adaptive learning mode utilizing machine learning algorithms to optimize system performance based on historical usage patterns.

9. The modular ventilation system of claim 8, wherein said control system further comprises:
wireless communication capabilities for remote monitoring and control;
data logging memory for storing operational parameters and environmental conditions; and
predictive maintenance algorithms generating service alerts based on component runtime and performance degradation.

10. The modular ventilation system of claim 1, wherein said control system is configured to execute ventilation cycles comprising:
scheduled operation based on time-of-day programming;
demand-based operation triggered by environmental thresholds; and
occupancy-based operation responsive to presence detection.

11. The modular ventilation system of claim 1, wherein said damper system comprises:
independently controllable either of the air intake dampers; and
failsafe spring-return mechanisms defaulting to predetermined positions upon power loss.

12. The modular ventilation system of claim 11, wherein said control system modulates damper positions to maintain a target indoor air quality threshold based on sensor inputs and runtime algorithms according to programmable settings.

13. The modular ventilation system of claim 1, further comprising:
adjustable bypass louvers enabling selective routing of airflow around said evaporator coil and said condenser coil;
a condensate pump system for active drainage management; and
a runtime log for monitoring filter loading status to generate notifications when filters require replacement.

14. The modular ventilation system of claim 1, further comprising maintenance-facilitating features including:
a control panel requiring a tool for removal to limit accessibility to electronic components, thereby providing a hindrance to protect users from damaging said components or risk of injury;
a tool-free filter access mechanism enabling service of said multi-stage air filtration assembly without tools;
color-coded and labelled electrical connectors with positive-lock mechanisms; and
machine-readable codes on serviceable components providing links to digital maintenance documentation.

15. The modular ventilation system of claim 1, wherein said sensor array further comprises:
at least one outdoor air temperature and humidity sensor;
at least one CO₂ sensor for indoor air quality monitoring;
at least one particulate matter sensor measuring PM2.5 concentrations; and
at least one indoor air temperature and humidity sensor.

16. A method of providing conditioned ventilation air, comprising:
continuously monitoring indoor and outdoor environmental parameters including temperature and humidity;
determining optimal ventilation requirements based on the monitored parameters and predetermined air quality thresholds;
selectively admitting outside air through motorized dampers when outdoor conditions are favorable;
filtering the air through multiple filtration stages to remove particulates and contaminants;
dehumidifying the air when humidity exceeds a setpoint using a modular dehumidification system;
sanitizing the air using UV-C germicidal irradiation and a photocatalytic oxidation (PCO) cell;
distributing the conditioned air to occupied spaces; and
automatically adjusting system operation based on real-time feedback to maintain target environmental conditions.

17. The method of claim 16, further comprising:
learning occupancy patterns over time; and
reducing ventilation rates during unoccupied periods while maintaining minimum air quality standards.

18. The method of claim 16, further comprising:
detecting filter loading through accumulated runtime log data;
generating maintenance alerts when filter replacement is needed; and
automatically adjusting airflow rate, dehumidification run time, and recirculation damper operation based on initial outside humidity and temperature readings.

19. The method of claim 16, wherein said dehumidifying step comprises:
cycling the dehumidification system based on both absolute humidity levels and dew point calculations; and
modulating H-Cell dehumidification run times to optimize moisture removal while minimizing energy use and overcooling.

20. An air purification module for integration with HVAC systems, comprising:
a housing configured for in-line duct mounting;
at least one UV-C germicidal irradiation light source;
a photocatalytic substrate positioned to receive UV-C radiation from said light source;
electrical interface connectors configured for activation and control by a system control PCB;
wherein said substrate catalyses oxidation of airborne organic compounds when irradiated.

21. The air purification module of claim 20, wherein:
said photocatalytic substrate comprises titanium dioxide nanoparticles deposited on a high-surface-area support matrix; and
said UV-C germicidal irradiation light source emits radiation in both UV-A and UV-C spectrums.

22. The air purification module of claim 21, further comprising:
a removable catalyst cartridge enabling periodic replacement; and
an hours-run meter tracking UV-C lamp usage.

23. The air purification module of claim 21, wherein said module is configured to be positioned downstream of mechanical filtration and upstream of supply air distribution to provide final-stage air treatment, and wherein said electrical interface connectors are configured for communication with an external system control PCB.
